(19) United States
(12) Patent Application Publication
(10) Pub. No.: US 2007/0072286 A1
(43) Pub. Date: Mar. 29, 2007

(54) Label-free detection of biomolecules

(75) Inventors: Joukje Garrelina Orsel, Delft (NL); Johannes Willem Hofstraat, Eindhoven (NL); Hendrik Roelof Stapert, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: Koninkljke Philips Electronics N.V., Eindhoven (NL)

(21) Appl. No.: 10/573,741

(22) PCT Filed: Sep. 20, 2004
(86) PCT No.: PCT/IB04/51794

§ 371 Date: Mar. 28, 2006

(30) Foreign Application Priority Data
Sep. 29, 2003 (EP) 03103597.5

(51) Int. Cl. C12M 1/34

(52) U.S. Cl. 435/287.2

(57) ABSTRACT

The invention provides a method and a device for label-free detection of biomolecules or analytes in a sample liquid. The method comprises the steps of allowing an analyte to bind to one of at least two conductive surfaces. An alternating electrical field between at least two conductive surfaces. Amplitude and phase of alternating current, flowing between the at least two conductive surfaces, are compared with amplitude and phase of reference signal. From the difference between both currents it is possible to determine whether an analyte is present at the conductive surface.

The present invention relates to a method and devices for the label free detection of biomolecules or other analytes utilizing the change in dielectric properties at a surface upon association of a biomolecule with that surface.

Detection of biomolecules is necessary in many applications such as medical diagnostics and monitoring of environmental and food safety. Commonly, biosensors comprise a surface on which target-specific recognition molecules are immobilized. This surface is brought into contact with a sample comprising one or more analytes of interest, which are allowed to bind and thus also become immobilized. These binding events then have to be translated into a measurable signal. To be able to detect biomolecules in a sensitive and specific manner, development steps and labels are usually applied. An example of development steps is the use of secondary, tertiary or even more antibodies, of which the first one binds the immobilized target and the last one carries a label. For other assays, such as DNA-microarrays, the targets are often first labeled before they are allowed to bind the recognition layer. Here, the labels used most are luminophores, but other examples of labels are radioactive isotopes and enzymes that can convert a substrate into a product that can be detected optically or electrically.

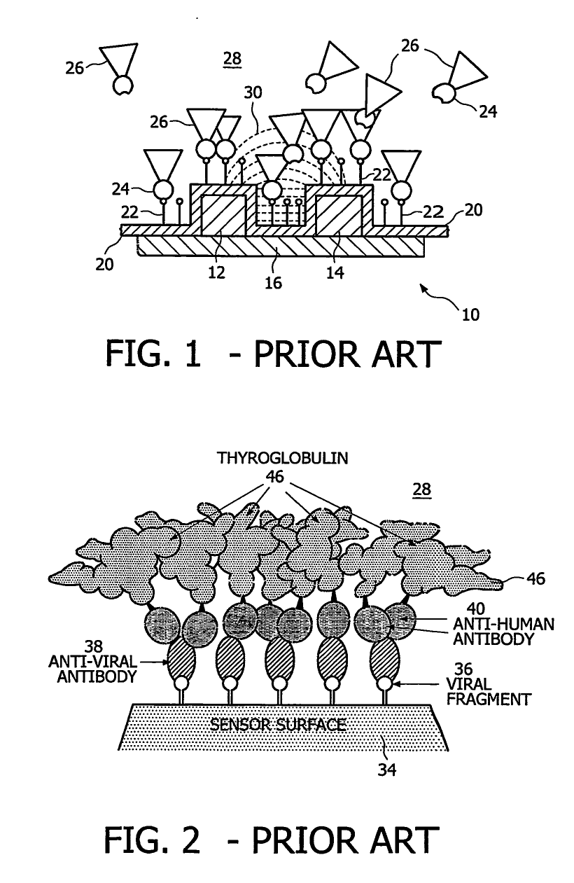

In U.S. Pat. No. 5,114,674 a capacitive affinity sensor is described. In a first embodiment, the sensor 10, which is illustrated in FIG. 1, comprises two electrodes 12, 14, which have opposite polarities, are positioned onto a base layer 16 and are insulated by a passivating layer 20. Receptors 22 extend from the passivating layer 20 and form a biochemically active layer. Each receptor 22 of this layer is a potential binding site for a molecule of a specific analyte 24. Large molecules 26 bind to the analyte 24 to form large molecular chains that bind to the receptor 22 as an added array in an electric field 30 between the electrodes 12, 14. These large molecular chains have low dielectric constants and displace a great amount of high dielectric constant solvent 28 from the electric field 30. The large molecular chains bind as an array that greatly increases the thickness of the dielectric material in the capacitive affinity sensor 10 and greatly changes the dielectric properties of that sensor 10.

In a second embodiment of the invention of U.S. Pat. No. 5,114,674 a capacitive affinity sensor using direct binding is described. The sensor, according to this second embodiment, is illustrated in FIG. 2. A sensor surface 34 comprises a base layer 16 and a passivating layer 20 as described in the first embodiment shown in FIG. 1. A viral fragment 36, which is an example of the receptor molecule 22 of the first embodiment, extends from the sensor surface 34 in a biochemical active layer. The analyte in the solvent 28 is a human anti-viral antibody 38. This human anti-viral antibody 38 is bio-specific to the viral fragment 36 to bind to that fragment 36. An anti-human antibody 40 and a bound protein molecule 46 are added to the solvent 28. The bound protein molecule 46 is bound to the anti-human anti-body 40 before both are added to the solvent 28. A number of anti-human antibodies 40 bind to each human anti-viral antibody 38, forming an array of large molecular chains. The molecular chains are very large, have low dielectric constants, and, therefore, displace a great amount of the solvent 28 which has a high dielectric constant. The dielectric properties of the sensor vary greatly with the concentration of the antiviral antibody 38 in the solvent 28.

In both embodiments, the moving low dielectric constant analyte molecule displaces higher dielectric constant solvent molecules from a biochemically active layer between the two electrodes, hereby reducing the capacitance between the two electrodes. The capacitance between the two electrodes is inversely proportional to the concentration of the analyte being measured by a sensor according to the invention of U.S. Pat. No. 5,114,674.

A disadvantage of the device of the above invention is that the analytes in the solvent require labeling and hence multiple development steps. The use of development steps and labels results in longer and more complex assays, increased use of expensive biomolecules and labels and more complicated and expensive assay devices. For fast and cost-effective measurements, the ideal assay is label-free. However, label-free assays hardly exist, because they usually lack the required sensitivity and specificity.

Also, the labeling of targets prior to their interaction with the recognition surface can cause changes in the target molecules, which may effect their efficacy, may change their concentration and/or hamper their accurate detection, for example when labeling varies from molecule to molecule or from measurement to measurement.

Furthermore, when multiple analytes are detected in one volume, the higher assay complexity caused by multiple development and labeling steps quickly leads to increased cross-reactivity and other background problems. Using a label-free assay would avoid the need of development steps and the disadvantages they cause for multiplexed detection.

It is an object of the present invention to provide a method and a device for the detection of one or more biomolecules, complexes of molecules or other analytes, which does not require the application of labels.

The above objective is accomplished by a method and a device according to the present invention.

The method provides a device for label-free detection of an analyte in a sample liquid. The device comprises at least two conductive surfaces between which an electric field can be applied, at least one of the conductive surfaces comprises immobilized target-specific affinity probes. The electric field is developed so as to be influenced by molecules which attach themselves to the immobilized target-specific affinity probes. Furthermore, the device comprises a means for providing an electrical field with a frequency between 10⁻² and 10⁶ Hz between the two conductive surfaces. The frequencies used for detection are chosen so as to detect interactions at the surface or in the dielectric interface formed at the surface. To enhance surface sensitivity, preferably, frequencies between 10⁻² and 10² Hz may be used.

Furthermore, the device may comprise a measuring means for measuring amplitude and/or phase of a first alternating current flowing between a first conductive surface with immobilized target-specific affinity probes and a second conductive surface which may or may not comprise the same target-specific affinity probes as the first conductive surface. Alternatively, an impedance measurement can be made.

The device of the present invention may furthermore comprise a comparator for comparing amplitude and phase of the first alternating current with amplitude and phase of a reference signal.

In a specific embodiment of the present invention, the device may comprise a first and a second conductive surface. The first conductive surface may comprise immobilized target-specific affinity probes on or at at least one side. The first and second conductive surfaces may be positioned substantially parallel to each other, the or a side immobilized with target-specific probes facing the second conductive surface.

In yet another embodiment, at least part of at least one conductive surface may interdigitate with at least part of at least one other conductive surface.

The analyte which may be determined using the method and device of the present invention may for example be a peptide, protein, antibody or a fragment thereof, enzyme, polynucleotide, oligonucleotide, carbohydrate, lipid, metabolite, cofactor, hormone, cytokine, cell, microorganism, virus, bacteria, algae, protozoa, drug, pesticide, herbicide, fungicide, toxin, vitamin, polysacchraide, a glycosilated site or any other small molecule or a combination of the aforementioned, for example a peptide comprising one or more carbohydrate groups or an enzyme with a bound cofactor.

The sample liquid which may be used in the invention may for example be an analytical solution, a bodily fluid such as blood, plasma, serum, urine, saliva, lung fluid or cerebrospinal fluid, a cell extract, waste water, any fluid in industrial processing, milk, drinking water, surface water or any other food product or solution thereof.

The target-specific affinity probe that may be used in the present invention may for example be a peptide, protein, antibody or a fragment thereof, enzyme, polynucleotide, oligonucleotide, aptamer, carbohydrate, oligosaccharide, lipid, metabolite, cofactor, hormone, cytokine, cell, microorganism, virus, drug, pesticide, herbicide, fungicide, toxin, vitamin or any other small molecule or a polymer having specific binding properties or a combination of the aforementioned, for example a peptide comprising one or more carbohydrate groups, an enzyme with a bound cofactor or a multimeric protein.

Furthermore, the present invention provides a method for label-free detection of an analyte in a sample liquid. The method comprises exposing at least one conductive surface to a sample liquid to allow association between the analyte in the sample liquid and at least one target-specific affinity probe on or at at least one conductive surface. In a next step of assaying of at least one conductive surface during any of the previous steps, an electrical field is applied between a first conductive surface with at least one immobilized target-specific affinity probe and a second conductive surface which may or may not comprise the same immobilized target-specific affinity probe, followed by measuring an electrical property of a resulting first alternating current such as amplitude and phase. The frequency of the applied field is between 10⁻² and 10⁶ Hz. Preferably, the frequency of the applied field may be between 10⁻² and 10² Hz In a next step the electrical property, e.g. amplitude and phase, of the first alternating current is compared with an electrical property, e.g. amplitude and phase, of a reference signal, thus generating a comparison result. From the comparison result, it is then determined whether an analyte has associated with at least one of the target-specific affinity probes.

In one embodiment of the present invention, the reference signal may be a calibration signal independently obtained at a conductive substrate similar to the at least one conductive surface that comprises immobilized target-specific affinity probes without incubation of an analyte.

In another embodiment, the method may furthermore comprise an assaying step of at least one conductive surface at which at least one target-specific affinity probe is immobilized before exposing to the liquid sample, which results in a second alternating current. This assaying step may comprise the same steps as the assaying step after exposing to the liquid sample. In this embodiment, the second alternating current may be the reference signal.

In still another embodiment the reference signal may be a set of measurements or frequency spectra.

The method of the present invention may furthermore comprise removing the sample liquid.

The method may further comprise rinsing the conductive surface with a washing solution to remove material that is non-specifically bound to an immobilized target-specific affinity probe.

In another embodiment, the method may furthermore comprise the step of rinsing the conductive surface to replace the sample liquid or the washing solution with a measurement solution.

In one embodiment of the present invention the step of applying an electrical field between the conductive surface with at least one immobilized target-specific affinity probe and a second conductive surface which may or may not comprise the same immobilized target-specific affinity probe and the step of measuring amplitude and phase of a first alternating current are repeated while varying the frequency of the alternating electrical field in order to obtain a dielectric spectrum.

The method may furthermore comprise a step of varying temperature and/or composition of the washing or measurement solution.

These and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference Figures quoted below refer to the attached drawings.

FIGS. 1 and 2 show a capacitive affinity sensor according to the prior art.

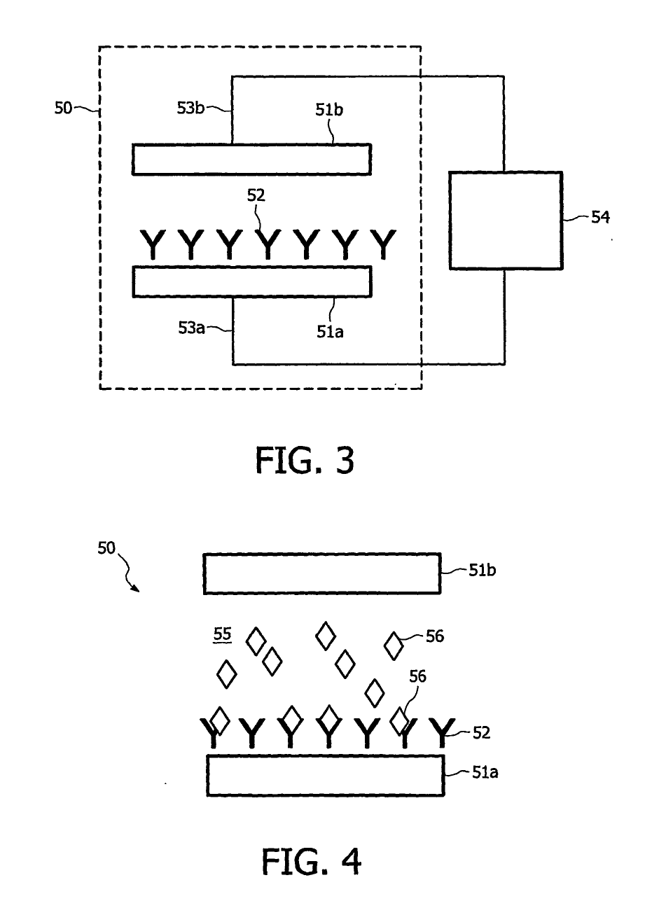

FIGS. 3 and 4 show a sensor device according to an embodiment of the present invention.

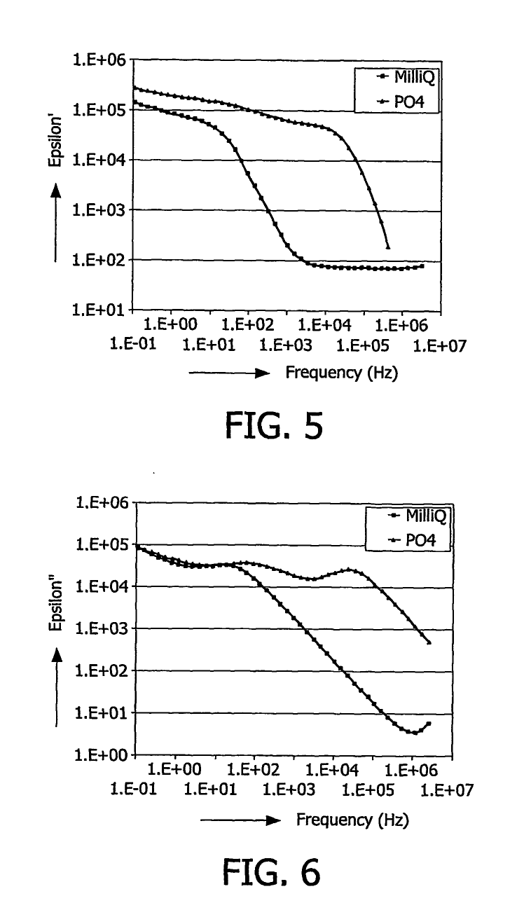

FIGS. 5-13 show measurement curves according to a specific example of the present invention.

In the different Figures, the same reference Figures refer to the same or analogous elements.

The present invention will be described with respect to particular embodiments and with reference to certain drawings but the invention is not limited thereto; it is limited only by the claims. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The present invention provides a method and a sensor device for the label-free detection of biomolecules or other analytes in a solvent utilizing the change in dielectric properties at a conductive surface upon association of the biomolecule or analyte with that surface or with an insulating layer on that surface.

The device 50 of the present invention may comprise at least two conductive surfaces 51a and 51b between which an electrical field can be applied, at least one of the surfaces 51a and 51b having immobilized target-specific affinity probes 52 attached thereto or attached to an insulating layer on the at least one surface, and conductive connectors 53a, 53b that allow for the connection to another device 54, either separate from or integrated in the sensor device 50 of the present invention, that generates the application of the electrical field and measures the amplitude and/or phase of the resulting current.

In FIG. 3 a device 50 according to one embodiment of the present invention is illustrated. The device may comprise two conductive surfaces 51a, 51b. The conductive surfaces 51a, 51b may for example comprise a metal (e.g. copper, gold, silver, platinum) a conductive metal oxide (e.g. indium tin oxide, indium zinc oxide) or a conductive polymer (e.g. polyaniline, polypyrrole, or poly(ethylenedioxothiophene)/polystyrene sulfonic acid blends). The conductive surfaces 51a, 51b may be massive, made of one conductive material, or may be a layer on a substrate, the substrate preferably being an insulator. When the conductive surfaces 51a, 51b are comprised of a conductive layer onto a substrate of a different material, the surface of the conductive layer may be at the same height as the surface of the substrate or alternatively it may be elevated or recessed in comparison with the surface of the substrate. The conductive surfaces 51a, 51b may have any useful shape and/or size and may be arranged in any useful configuration. Shapes may be any suitable shape such as a circle, a polygon, a triangle, a rectangle and a strip, the strip being either straight or comprising bends or corners, or two or more of these shapes linked together, for example in the shape of an interdigitated structure. The size of the conductive surfaces 51a, 51b may be anything between 2 cm in diameter or length or width and 1 nm in diameter or length or width. The conductive surfaces may be flat or comprise recessed or elevated regions.

At least one of the conductive surfaces 51a, 51b may have immobilized target-specific affinity probes 52. In this embodiment of the invention only conductor surface 51a is immobilized with target-specific affinity probes 52. In the further description of this embodiment the conductive surface immobilized with target-specific affinity probes 52 will be denoted as conductive surface 51a, the other conductive surface will be denoted as conductive surface 51b. Preferably, the target-specific affinity probe 52 may be any suitable probe, such as a peptide, a protein, an antibody or a fragment thereof, an enzyme, a polynucleotide, an oligonucleotide, an aptamer, a carbohydrate, an oligosaccharide, a lipid, a metabolite, a cofactor, a hormone, a cytokine, a cell, a microorganism, a virus, a drug, a pesticide, a herbicide, a fungicide, a toxin, a vitamin or any other small molecule or a combination of the aforementioned, for example a peptide comprising one or more carbohydrate groups, an enzyme with a bound cofactor or a multimeric protein. Most preferably, the target-specific affinity probe 52 may be an antibody or a fragment thereof, an aptamer, an oligosaccharide or an oligonucleotide.

The target-specific affinity probe 52 may be deposited onto the conductive surface 51a by for example flow, dropping, spotting, contacting or by any other suitable deposition technique.

Immobilization of the target-specific affinity probes 52 onto the conductive surface 51a may be achieved in different ways.

In one embodiment, the conductive surface 51a may be modified before immobilization of the target-specific affinity probes 52. Possible modifications may include the conferring of active groups to the conductive surface 51a such as carboxylic groups, amine groups and the like and/or the activation of these groups such as the formation of a succinimidyl ester. For many purposes, the conductive surface 51a may be modified with alkyl chains or modifications thereof. The alkyl chains may comprise active groups which may be activated and used for the binding of other alkyl chains or modifications thereof, for the binding of affinity probes 52 or for the binding of molecules or complexes of molecules that can be used for the immobilization of affinity probes 52. The conductive surface may be coated with a self organizing monolayer or SAM.

In another embodiment, the target-specific affinity probes 52 may be modified to comprise one or more active groups that may be used for their immobilization, such as for example, but not limited to, carboxylic acid groups, amine groups, hydroxyl groups, epoxy groups, isocyanates, (meth)acrylates, thiols, sulfides, biotin, affinity peptides, oligosaccharides, oligonucleotides or polynucleotides and activated esters such as succinimidyl esters.

Target-specific affinity probes 52 may adsorb or bind to the conductive surface 51a directly, an example of binding being the association of affinity probes 52 comprising a thiol group with a gold, silver or platinum surface.

Alternatively, affinity probes 52 may bind to active or activated groups that have been conferred to the conductive surface 51a. An active or activated group is connected with the conductive surface 51a via chemical bonds. The number of chemical bonds may be between one and one hundred thousand and may be any number in-between. When more than one chemical bond is present between conductive surface 51a and the active or activated groups used for immobilization of the affinity probe 52, one can speak of a linker. Linker molecules may have any composition. Frequently used molecules may be alkyl chains and modifications thereof, ethyleneglycol chains and hydrogels.

Another mode of immobilization of an affinity probe 52 is via association with one or more molecules or molecule complexes that specifically bind the affinity probe 52 and that have already been immobilized to the conductive surface 51a. Non-limiting examples are the association of a biotinylated peptide with surface immobilized streptavidin, the association of an antibody with another antibody that is immobilized and is an anti-immunoglobulin and the association of an oligonucleotide with a second oligonucleotide that has been immobilized, part of the first oligonucleotide being complementary to at least part of the second oligonucleotide.

The configuration of the two conductive surfaces 51a, 51bmay be substantially in parallel, the side 51a with immobilized affinity probes 52 facing the other conductive surface 51b. Alternatively, the two surfaces 51a , 51b may not be completely in parallel with regard to each other, but be placed at any angle between 0 and 180 degrees. Parts of one conductive surface 51a, 51b may interdigitate with parts of the other conductive surface 51a, 51b. The spacing between the two conductive surfaces 51a, 51b may vary between 1 nanometer and 1 centimeter.

After immobilizing the target-specific affinity probe 52 onto the conductive surface 51a an optional step of assaying the conductive surface 51a may be performed before the device 50 is exposed to a sample liquid 55 comprising an analyte 56 which has to be analyzed. Then, the conductive surfaces 51a, 51b are exposed to the sample liquid 55 to allow association between a possibly present analyte 56 and the affinity probe 52 (see FIG. 4).

Preferably, the sample liquid 55 may be an analytical solution, a bodily fluid such as blood, plasma, serum, urine, saliva, lung fluid or cerebrospinal fluid, a cell extract, waste water, any fluid in industrial processing, milk, drinking water, surface water or any other food product or solution thereof.

Preferably, the analyte 56 may be a peptide, a protein, an antibody or a fragment thereof, an enzyme, a polynucleotide, an oligonucleotide, a carbohydrate, a lipid, a metabolite, a cofactor, a hormone, a cytokine, a cell, organelles of a cell, cell lysates, cell membrane, a micro-organism, a virus, bacteria, protozoa, algae, a drug, a pesticide, a herbicide, a fungicide, a toxin, a vitamin or any other small molecule or a combination of the aforementioned, for example a peptide comprising one or more carbohydrate groups or an enzyme with a bound cofactor. More preferably, the analyte 56 may be a protein or a polynucleotide.

In a next step, the sample liquid 55 may optionally be removed, followed by an optional rinsing step of the surface 51a with a washing solution to remove non-specifically bound material. Washing solutions may for example comprise different salts at different concentrations, sugars, detergents like e.g. Tween or anything else to remove non-specific binding. If used during measurement the washing solution may also comprise additional components or optimized concentrations that may enhance the signal during measurement and/or it may comprise compounds that can easily be seen in the dielectric spectrum (preferably at higher frequencies) and can be used for characterizing the extent of the washing step. Other methods of removing non-specifically bound material may be used, e.g. application of electric or magnetic fields, raising the temperature, etc.

In a next optional step the surface 51a may be rinsed again to replace the sample liquid 55 or the washing solution with a measurement solution. The measurement solution may comprise a certain salt type and concentration, certain buffer salts, e.g. large zwitterions, sugars, detergents, etc.

A step of assaying the surface 51a for the presence of the analyte 56 may be performed sequentially or simultaneously during any of the preceding optional steps.

The steps of assaying the conductive surface 51a before and/or after exposing to the sample liquid 55 may be performed according to the following steps. In a first step an alternating electrical field is applied, by applying a voltage between the conductive surface 51a with immobilized affinity probes 52 and the conductive surface 51b.

The frequency of the applied alternating electrical field may lie between 10⁻³ and 10 ¹² Hz, but to enhance surface sensitivity, preferably lies between 10⁻³ and 10⁷ Hz, more preferably lies between 10⁻² and 10⁶ Hz, and most preferably lies between 10⁻² and 10² Hz. The amplitude of the applied alternating electrical field may preferably be between 0 and 10 V, more preferably between 0.001 and 1 V, and most preferably may be between 0.01 and 0.2 V.

Then, the amplitude and phase of a resulting alternating current are measured. From the amplitude and phase of said alternating current, information may be obtained pertaining to the dielectric properties of the material under analysis, such as its dielectric constant or impedance. From the component of the current that is in phase with the voltage the conductive part of the dielectric constant may be deduced, while from the component of the current that is out of phase with the electrical field, the capacitive part of the dielectric constant may be obtained. From the current signal, many other quantities may be obtained, such as the capacitance and the impedance.

Optionally, the steps of applying an electrical field and measuring amplitude and phase of a resulting current may be repeated while varying the frequency in order to obtain a dielectric spectrum, through which the amount of information that can be obtained may be increased.

In a next optional step, the foregoing steps may be repeated while varying one or more parameters, such as e.g. the temperature or the composition of the washing or measurement solutions.

Next, a step of comparing, sequentially or simultaneously during any of the preceding steps, the amplitude and phase of the alternating current, or quantities or values calculated therefrom, after exposure of the conductive surface 51a to the analyte 56, with the amplitude and phase of a reference signal. For the comparison a single measurement may be used, but also a set of measurements and complete frequency spectra.

The reference signal may be determined in different ways. In one embodiment, the reference signal may be a calibration signal which may be obtained before and independent of performing the measurement with the sensor device 50 at a conductive substrate similar to the conductive surface 51a.

In another embodiment, the reference signal is obtained in the sensor device 50 at the conductive surface 51a before exposing the device 50 to the sample liquid 55.

In a further embodiment, instead of using for a comparison the assessment of the same conductive surface 51a before association of the analyte 56 with the affinity probe 52, the assessment of the other conductive surface 51b that was not exposed to the analyte 56 may be used.

From the change in amplitude and phase of the alternating current, or any value derived therefrom, after association of the analyte 56 with the target-specific affinity probe 52, the presence of the analyte 56 can be deduced. Furthermore, the amount of analyte 56 units may be determined qualitatively or the number of analyte 56 units may be determined in a quantitative manner.

For accurate comparison of the spectra before, during and after binding it is important to use data obtained in the same measurement solution, which require washing steps. The salt concentration of the measurement solution may be checked using the dielectric spectra at other (preferably higher) frequencies and can be used as a control for the extent of the washing step, and thus the accuracy of the analyte 56 concentration determination.

Additionally or alternatively, correction for remaining salts not washed away in the development procedure may be achieved by application of measurements at other frequencies in the dielectric spectrum than those used for analyte 56 detection.

In another embodiment of the invention, the method of the invention may be used to detect multiple targets. This may be achieved by placing multiple conductive surfaces 51a, 51b, on each of which different target-specific affinity probes 52 are immobilized, at a certain distance and exposing them at the same time to one and the same volume of sample liquid 55. The conductive surfaces may then be assessed before and after exposure to the sample liquid 55 either in parallel or sequentially. In that way, it is possible to analyze sample liquids 55 that comprise different analytes 56 with one measurement using the device 50 of the present invention.

In a specific example, the device 50 of the present invention is applied to investigate a sample liquid 55 comprising 1 picomolar (pM) concentration of von Willebrand Factor (vWF), which is a blood clotting substance, as an analyte 56 in 10 mM phosphate buffer. The conductive surfaces (51a, 51b) are interdigitated gold electrodes on which different self assembled monolayers (SAMs) were formed on which anti-vWF antibodies were immobilized.

First, in FIG. 5 the real or capacitive part of the dielectric constant is plotted as a function of frequency for MilliQ (deionised water) and 10 mM phosphate buffer, in order to show the change with frequency when measuring in the bulk or closer to the surface of interdigitated gold electrodes with a spacing of 10 micrometers. At low frequencies more ions are in the bulk of the sample liquid. Hence, the electric double layer becomes thinner and the capacity becomes higher. In FIG. 5 it can be seen that at lower frequencies the capacitive part of the dielectric constant is higher that at high frequencies. In the MilliQ solution, fewer ions are present and hence the capacitive part of the dielectric constant is higher than at the same frequency in the phosphate buffer solution. FIG. 6 shows the imaginary or conductive part of the dielectric constant

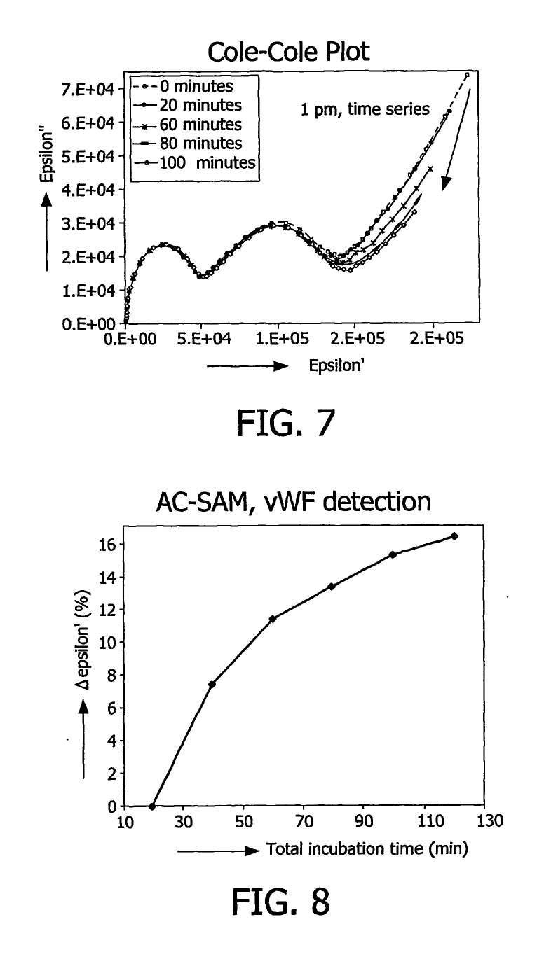

In FIG. 7 a Cole-Cole plot of the sensor is shown after multiple injections of 1 pM concentration of vWF in 10 mM phosphate buffer. Different injections are performed within a time period of 20 minutes. Between each injection the sensor is treated with a washing solution. The Figure shows the conductive part of the dielectric constant as a function of the capacitive part.

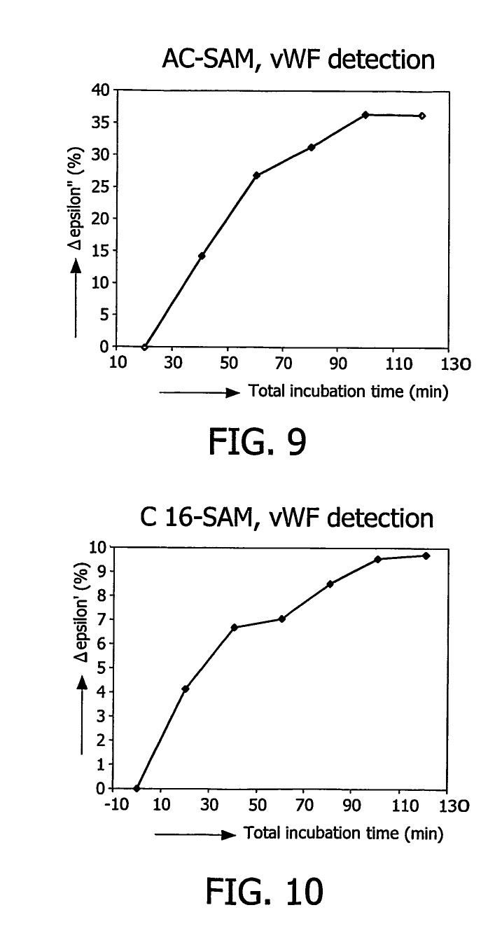

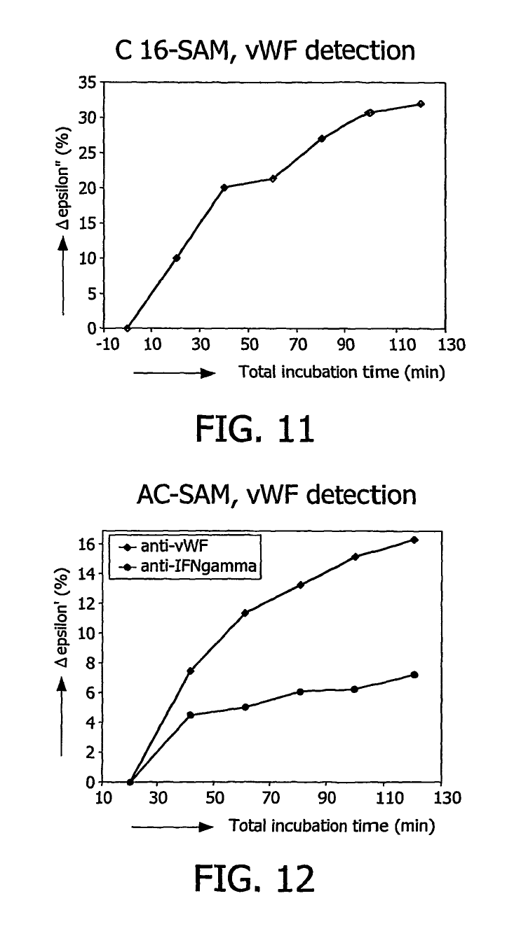

FIG. 8 to 11 illustrate that it is possible to detect an analyte in a sample liquid by applying the device and method of the present invention. In FIGS. 8 and 9 the interdigitated gold electrodes are covered with a SAM of acetylcysteine. Measurements are performed at a frequency of 0.1 Hz. FIGS. 10 and 11 show results obtained at interdigitated gold electrodes which are covered with a SAM of mercaptohexadecanoic acid. Again, measurements are performed at 0.1 Hz.

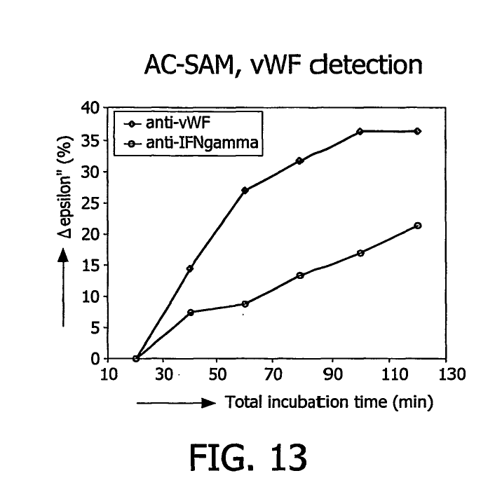

FIGS. 12 and 13 resp. show the capacitive and conductive part of the dielectric constant as a function of total incubation time measured at interdigitated gold electrodes which are immobilized with anti-vWF (upper curve) or with anti-IFNgamma (lower curve). Anti-IFNgamma is non-specific for vWF. From these results it becomes clear that at least part of the signal is specific if the results from FIGS. 8 to 11 are compared with the detection of non-specific binding of a 1 pM concentration of vWF on a SAM on which anti-interferon gamma antibody is immobilized. The difference between the upper and lower curve in FIGS. 12 and 13 represents the specific part of the signal.

With the method and device 50 of the present invention, analytes 56 which have to be determined do not have to be labeled, as is the case in the prior art. For this purpose, the frequencies used for detection are chosen so as to detect interactions at the surface 51a or in the dielectric interface formed at the surface 51a. The impact of constituents in the sample liquid 55 consequently is strongly diminished, resulting in drastic improvement of the sensitivity (through reduction of interfering background signals) and of the specificity of the detection method. To increase the reliability of the quantitative measurement of an analyte 56 concentration, redundancy may be build in by using more than one sensor device 50 for the same analyte 56.

It is important to be noted that the method and device 50 of the present invention may not only be used for the measurement of biomolecules or complexes of molecules in an aqueous solution, but may also be applied for the detection of any other molecule or complex of molecules in any other kind of solution.

It is to be understood that although preferred embodiments, specific constructions and configurations, as well as materials, have been discussed herein for devices according to the present invention, various changes or modifications in form and detail may be made without departing from the scope and spirit of this invention.

1. A device (50) for label-free detection of an analyte (56) in a sample liquid (55), the device (50) comprising:
at least two conductive surfaces (51a, 51b), at least one of the conductive surfaces (51a, 51b) comprising immobilized target-specific affinity probes (52), and
means for providing an electrical field with a frequency between 10⁻² and 10⁶ Hz between the two conductive surfaces (51a, 51b).

2. A device (50) according to claim 1, furthermore comprising a measuring means for measuring amplitude and phase of a first alternating current flowing between a first conductive surface with immobilized target-specific affinity probes and a second conductive surface.

3. A device (50) according to claim 2, furthermore comprising a comparator for comparing amplitude and phase of the first alternating current with amplitude and phase of a reference signal.

4. A device (50) according to claim 1, comprising a first (51a) and a second conductive surface (51b), said first conductive surface (51a) comprising immobilized target-specific affinity probes (52) at at least one side.

5. A device (50) according to claim 1, wherein said first (51a) and said second conductive surface (51b) are positioned substantially parallel to each other, the or a side immobilized with target-specific probes (52) facing said second conductive surface (51b).

6. A device (50) according to claim 4, wherein at least part of conductive surface (51a) interdigitates with at least part of the conductive surface (51b).

7. A device (50) according to claim 1, wherein said analyte (56) is selected from the group consisting of a peptide, protein, antibody or a fragment thereof, enzyme, polynucleotide, oligonucleotide, carbohydrate, lipid, metabolite, cofactor, hormone, cytokine, cell, microorganism, virus, drug, pesticide, herbicide, fungicide, toxin, vitamin or any other small molecule or a combination of the aforementioned, for example a peptide comprising one or more carbohydrate groups or an enzyme with a bound cofactor.

8. A device (50) according to claim 1, wherein the sample liquid (55) is selected from a group consisting of an analytical solution, a bodily fluid such as blood, plasma, serum, urine, saliva, lung fluid or cerebrospinal fluid, a cell extract, waste water, any fluid in industrial processing, milk, drinking water, surface water or any other food product or solution thereof.

9. A device (50) according to claim 1, wherein the target-specific affinity probe (52) is selected from a group consisting of a peptide, protein, antibody or a fragment thereof, enzyme, polynucleotide, oligonucleotide, aptamer, carbohydrate, oligosaccharide, lipid, metabolite, cofactor, hormone, cytokine, cell, microorganism, virus, drug, pesticide, herbicide, fungicide, toxin, vitamin or any other small molecule or a combination of the aforementioned, for example a peptide comprising one or more carbohydrate groups, an enzyme with a bound cofactor or a multimeric protein.

10. A method for label-free detection of an analyte (56) in a sample liquid (55), the method comprising:
exposing at least one conductive surface (51a, 51b) with at least one target-specific affinity probe (52) immobilized thereon to a sample liquid (55) to allow association between said analyte (56) in said sample liquid (55) and at least one target-specific affinity probe (52),
assaying said at least one conductive surface (51a,51b) for the presence of the associated analyte (56), said assaying comprising:
applying an alternating electrical field between a first of at least one conductive surface (51a) and a second conductive surface (51b) thus generating a first alternating current flowing between said first (51a) and said second conductive surface (51b), the applied electrical field having a frequency between 10⁻² and 10⁶ Hz,
measuring an electrical property of said first alternating current,
comparing, sequentially or simultaneously during any of the preceding steps the measured electrical property of said first alternating current with an electrical property of a reference signal, thus generating a comparison result,
determining from the comparison result whether analyte (56) has associated with at least one of the target-specific affinity probes (52).

11. A method according to claim 10, wherein the comparing step includes comparing
amplitude and phase of said first alternating current with amplitude and phase of the reference signal.

12. A method according to claim 10, wherein the electrical field has a frequency between 10⁻² and 10² Hz.

13. A method according to claim 10, wherein said second conductive surface (51b) comprises the same target-specific affinity probes (52) as the first conductive surface (51a).

14. A method according to claim 10, wherein said reference signal is a calibration signal independently obtained using a conductive surface similar to said at least one conductive surface (51a, 51b) without incubation of an analyte (56).

15. A method according to claim 10, the method furthermore comprising assaying said at least one conductive surface (51a, 51b) at which at least one target-specific affinity probe (52) is immobilized before exposing to the liquid sample (55), resulting in second alternating current.

16. A method according to claim 15, wherein said reference signal is said second alternating current.

17. A method according to claim 10, furthermore comprising removing the sample liquid (55).

18. A method according to claim 10, furthermore comprising rinsing the conductive surface (51a, 51b) with a washing solution to remove material that is non-specifically bound to an immobilized target-specific affinity probe (52).

19. A method according to claim 10, furthermore comprising rinsing the conductive surface (51a, 51b) to replace the sample liquid (55) or the washing solution with a measurement solution.

20. A method according to claim 10, wherein applying an electrical field between the first conductive surface (51a) with at least one immobilized target-specific affinity probe (52) and the second conductive surface (51a, 51b) and measuring amplitude and phase of a first alternating current are repeated while varying the frequency of the alternating electrical field in order to obtain a dielectric spectrum.

21. A method according to claim 20, furthermore comprising varying temperature and/or composition of the washing or measurement solution.

22. A method according to claim 10, wherein the reference signal is a set of measurements or frequency spectra.
